(19) United States
(12) Patent Application Publication
(10) Pub. No.: US 2025/0017993 A1
(43) Pub. Date: Jan. 16, 2025

(54) FEED ADDITIVE, LAYING HEN FEED, AND METHOD FOR PRODUCING ASTAXANTHIN EGGS

(71) Applicant: Algae Origin Inc., Newton, MA (US)

(72) Inventor: Bingchun CHANG, Newton, MA (US)

(73) Assignee: Algae Origin Inc., Newton, MA (US)

(21) Appl. No.: 18/219,723

(22) Filed: Jul. 10, 2023

(51) Int. Cl. A61K 36/05; A23K 10/16; A23K 10/30; A23K 20/158; A23K 20/174; A23K 40/00; A23K 50/75; A61K 9/00; A61K 47/22; A61K 47/44

(52) U.S. Cl. CPC A61K 36/05; A23K 10/16; A23K 10/30; A23K 20/158; A23K 20/174; A23K 40/00; A23K 50/75; A61K 9/0053; A61K 47/22; A61K 47/44

(57) ABSTRACT

A feed additive, a laying hen feed, and a method for producing astaxanthin eggs are provided. The feed additive provided includes the following components in parts by weight: 5-50 parts of Haematococcus pluvialis, 6-10 parts of vegetable oil, and 6-8 parts of vitamin E. The chicken feed provided by the present invention includes the feed additive and a basal feed. The present invention can improve the enrichment of astaxanthin in Haematococcus pluvialis in the yolk, increase the pigment content of the yolk, and improve the nutrition value of egg products. According to the present invention, the feed additive described above with a specific content is added to the chicken feed, and the feed additive is fed to the hens at a specific feeding amount, so that the palatability of the feed and hens' intake of the feed are not influenced.

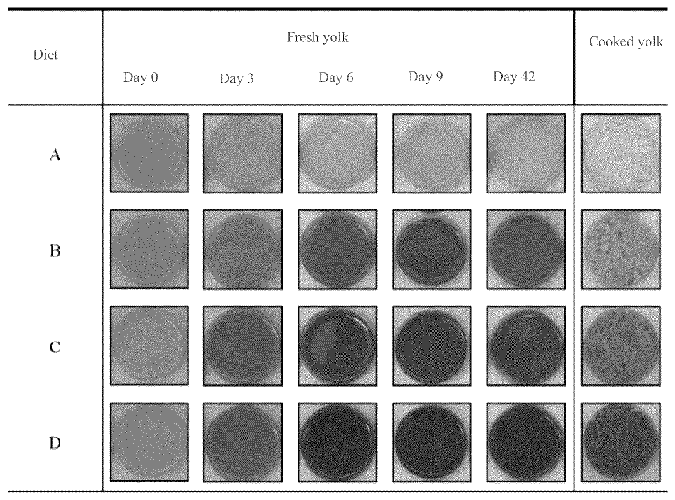

### TECHNICAL FIELD

The present invention belongs to the field of the preparation of feed additives, and particularly relates to a feed additive, a laying hen feed, and a method for producing astaxanthin eggs.

### BACKGROUND

Astaxanthin, a ketocarotenoid with a chemical name of 3,3′-dihydroxy-4,4′-diketo-β,β′-carotene, is a red solid powder, has liposolubility, is insoluble in water, and is soluble in an organic solvent. Adding astaxanthin to the poultry feed can increase the pigment content of egg yolks and promote health of laying hens. Astaxanthin has been used for many years to alter the color of yolks to achieve a desired color. Astaxanthin can promote health of hens by stimulating secondary antibodies of virus infection, and also has an anti-inflammatory effect.

Haematococcus pluvialis is a unicellular green alga which is an extremely ideal raw material for producing natural astaxanthin. Haematococcus pluvialis is the only natural astaxanthin-producing algae strain authorized for human consumption. Astaxanthin from Haematococcus pluvialis has the highest bioavailability in animals. The Haematococcus pluvialis algae powder has been approved in Japan as a natural food colorant and a natural pigment additive for fishing bait, and also has been approved by the European Commission as a food colorant for use in the food and beverage industries. Canada has also approved Haematococcus pluvialis as a natural red pigment component in salmon bait. U.S. Food and Drug Administration (FDA) has also approved Haematococcus pluvialis as a natural pigment additive in salmon bait and has explicitly prescribed Haematococcus pluvialis as a supplemental component to foods on the market.

The astaxanthin applied in the livestock and poultry cultivation at the present stage is industrially synthesized or industrially extracted. The quality and the content of astaxanthin need to be improved. It is unfavorable for organisms to completely absorb and utilize the astaxanthin. The astaxanthin content in the products obtained by the livestock and poultry cultivation is low.

### SUMMARY

In view of this, the present invention provides a feed additive, a laying hen feed, and a method for producing astaxanthin eggs. The present invention, by adding natural Haematococcus pluvialis, vegetable oil, and vitamin E to a feed additive, increases the content of natural astaxanthin in the feed. By feeding the laying hen feed provided by the present invention, the nutrition value and chromaticity of the eggs can be effectively improved and high-quality astaxanthin eggs can be obtained.

The present invention provides a feed additive including the following components in parts by weight: 5-50 parts of Haematococcus pluvialis, 6-10 parts of vegetable oil, and 6-8 parts of vitamin E.

Preferably, the Haematococcus pluvialis is a cell wall-broken Haematococcus pluvialis.

Preferably, the Haematococcus pluvialis is a dry algae powder of the cell wall-broken Haematococcus pluvialis or an algae sludge of the cell wall-broken Haematococcus pluvialis.

Preferably, the Haematococcus pluvialis is 25-35 parts by weight.

The present invention provides a chicken feed including the feed additive and a basal feed.

Preferably, the mass percentage of the feed additive in the chicken feed is 0.5-5%; the mass percentage of the basal feed is 93.4%-97.9%.

Preferably, the basal feed includes corn, soybean meal, stone powder, and a premix; the mass ratio of the corn, the soybean meal, the stone powder, and the premix is 60-61.6:24.9-25.5:7-7.2:3.5-3.6.

The present invention provides a method for preparing the chicken feed, including the following steps: pulverizing and mixing raw materials of the basal feed, and then mixing with the feed additive to obtain the chicken feed.

The present invention provides a method for producing astaxanthin eggs, including the following step: feeding laying hens with the chicken feed.

Preferably, the feeding amount of the chicken feed is 0.05-0.15 g/day; the feeding time of the chicken feed is 3-42 days.

Compared with the prior art, the present invention has the following beneficial effects: the feed additive provided by the present invention includes 5-50 parts of Haematococcus pluvialis, 6-10 parts of vegetable oil, and 6-8 parts of vitamin E; the three components act together, which can improve the enrichment of astaxanthin in Haematococcus pluvialis in the yolk, increase the pigment content of the yolk, and improve the nutrition value of egg products. The vegetable oil can effectively dissolve astaxanthin so that the astaxanthin is more easily absorbed by the chickens; the vitamin E can effectively prevent the astaxanthin from being rapidly oxidized and maintain the active state of the astaxanthin.

According to the present invention, the feed additive described above with a specific content is added to the chicken feed, and the feed additive is fed to the hens at a specific feeding amount, so that the palatability of the feed and hens' intake of the feed are not influenced, the hens' intake and the enrichment of astaxanthin can be promoted, and the resistance of the hens is improved.

After the intake of the feed including astaxanthin provided by the present invention by hens, astaxanthin and enzymes are released together and then absorbed by the small intestine. Free astaxanthin is emulsified into oil droplets and delivered to the liver, incorporated into very low-density lipoprotein LDL by the liver and then transported to the yolk for enrichment to form eggs rich in astaxanthin.

### BRIEF DESCRIPTION OF THE DRAWING

FIGURE is a color comparison of astaxanthin egg yolks (fresh and cooked state) obtained from feeding with different amounts of Haematococcus pluvialis, wherein group A has no Haematococcus pluvialis added; group B has 0.5% Haematococcus pluvialis added; group C has 1.5% Haematococcus pluvialis added; group D has 3.0% Haematococcus pluvialis added.

### DETAILED DESCRIPTION OF THE EMBODIMENTS

The present invention provides a feed additive including the following components in parts by weight: 5-50 parts of Haematococcus pluvialis, 6-10 parts of vegetable oil, and 6-8 parts of vitamin E.

In the present invention, the feed additive includes 5-50 parts of Haematococcus pluvialis, preferably 10-40 parts, and further preferably 25-35 parts. In the present invention, the Haematococcus pluvialis is preferably a cell wall-broken Haematococcus pluvialis, and further preferably a dry algae powder of the cell wall-broken Haematococcus pluvialis or an algae sludge of the cell wall-broken Haematococcus pluvialis. In the present invention, a method for preparing the dry algae powder of the cell wall-broken Haematococcus pluvialis preferably includes the following steps: culturing and inducing the Haematococcus pluvialis to turn red in a closed pipeline glass photoreactor, centrifuging and collecting the algae sludge, freeze-drying the algae sludge into powder, and breaking the cell wall for later use. In the present invention, a method for preparing the algae sludge of the cell wall-broken Haematococcus pluvialis preferably includes the following steps: culturing and inducing the Haematococcus pluvialis to turn red in a closed pipeline glass photoreactor, centrifuging and collecting the algae sludge, and breaking the cell wall for later use.

In the present invention, the feed additive includes 6-10 parts of vegetable oil, preferably 7-9 parts, and more preferably 8.9 parts. In the present invention, the kind of the vegetable oil is preferably soybean oil. The vegetable oil can effectively dissolve astaxanthin so that the astaxanthin is more easily absorbed by the chickens, and meanwhile, the energy content of the feed additive is improved.

In the present invention, the feed additive includes 6-8 parts of vitamin E, preferably 6.5-7.5 parts, and more preferably 7.1 parts. In the present invention, the vitamin E is used for effectively preventing the astaxanthin from being rapidly oxidized, maintaining the active state of the astaxanthin, and simultaneously promoting hormone secretion, improving reproductive ability, and enhancing the resistance of organisms to diseases.

The present invention further provides a chicken feed including the feed additive and a basal feed.

In the present invention, the mass percentage of the feed additive in the chicken feed is preferably 0.5-5%, more preferably 3-4.9%, and most preferably 4.6%; the mass percentage of the basal feed is preferably 93.4%-97.9%, more preferably 94%-95%, and most preferably 94.4%.

In the present invention, the basal feed preferably includes corn, soybean meal, stone powder, and a premix; the mass ratio of the corn, the soybean meal, the stone powder, and the premix is preferably 60-61.6:24.9-25.5:7-7.2:3.5-3.6. In the present invention, the corn, the soybean meal, the stone powder, and the premix are all commercially available products; the premix is preferably purchased from Beijing Dabeinong Technology Group Ltd.

The present invention provides a method for preparing the chicken feed, including the following steps: pulverizing and mixing raw materials of the basal feed, and then mixing with the feed additive to obtain the chicken feed.

In the present invention, the pulverization is preferably carried out by physical mechanical pulverization, and the particle size of the pulverized raw material is preferably 10 mm.

The present invention further provides a method for producing astaxanthin eggs, including the following step: feeding laying hens with the chicken feed.

In the present invention, the feeding amount of the chicken feed is preferably 0.05-0.15 g/day, more preferably 0.08-0.12 g/day, and most preferably 0.1 g/day; the feeding time of the chicken feed is preferably 3-42 days, and more preferably 4-30 days.

The technical solutions provided by the present invention will be described in detail below with reference to the examples, which, however, should not be construed as limiting the protection scope of the present invention.

The method for preparing the Haematococcus pluvialis algae powder: filtering and collecting the algae sludge of Haematococcus pluvialis using a plate filter press, adding the algae sludge into a freeze dryer for drying, adding the dried algae powder into a blender for cell wall breaking and detecting that the cell wall breaking rate reaches 90% or more to obtain the Haematococcus pluvialis algae powder.

The method for preparing the algae sludge of Haematococcus pluvialis: filtering and collecting the algae sludge of Haematococcus pluvialis using a plate filter press, adding the algae sludge into a blender for cell wall breaking and detecting that the cell wall breaking rate reaches 90% or more to obtain the algae sludge of Haematococcus pluvialis, measuring the water content of the cell wall-broken algae sludge, adding the algae sludge at an addition amount calculated according to the addition amount of the dry powder feed in proportion.

### Example 1

The feed additive: 0.5 kg Haematococcus pluvialis algae powder, 0.89 kg vegetable oil, and 0.71 kg vitamin E.
The basal feed:
61.6 kg corn, 25.5 kg soybean meal, 7.2 kg stone powder, and 3.6 kg premix.
The preparation method: mixing and pulverizing the corn, the soybean meal, the stone powder, and the premix, and then mixing the mixture with the Haematococcus pluvialis algae powder, the vegetable oil, and the vitamin E.

### Example 2

The feed additive: 1.5 kg Haematococcus pluvialis algae powder, 0.89 kg vegetable oil, and 0.71 kg vitamin E.
The basal feed:
61 kg corn, 25.1 kg soybean meal, 7.2 kg stone powder, and 3.6 kg premix.
The preparation method: mixing and pulverizing the corn, the soybean meal, the stone powder, and the premix, and then mixing the mixture with the Haematococcus pluvialis algae powder, the vegetable oil, and the vitamin E.

### Example 3

The feed additive: 3 kg Haematococcus pluvialis algae powder, 0.89 kg vegetable oil, and 0.71 kg vitamin E.
The basal feed:
60 kg corn, 24.9 kg soybean meal, 7.0 kg stone powder, and 3.5 kg premix.
The preparation method: mixing and pulverizing the corn, the soybean meal, the stone powder, and the premix, and then mixing the mixture with the Haematococcus pluvialis algae powder, the vegetable oil, and the vitamin E.

### Comparative Example 1

The feed additive: 3 kg Haematococcus pluvialis algae powder.
The basal feed:
61.6 kg corn, 25.5 kg soybean meal, 7.2 kg stone powder, and 3.6 kg premix.
The preparation method: mixing and pulverizing the corn, the soybean meal, the stone powder, and the premix, and then mixing the mixture with the Haematococcus pluvialis algae powder.

### Comparative Example 2

The feed additive: 0.89 kg vegetable oil and 0.71 kg vitamin E.
The basal feed:
61.6 kg corn, 25.5 kg soybean meal, 7.2 kg stone powder, and 3.6 kg premix.
The preparation method: mixing and pulverizing the corn, the soybean meal, the stone powder, and the premix, and then mixing the mixture with the vegetable oil and the vitamin E.

### Comparative Example 3

The feed additive: 3 kg Haematococcus pluvialis algae powder and 0.71 kg vitamin E.
The basal feed:
60 kg corn, 24.9 kg soybean meal, 7.0 kg stone powder, and 3.5 kg premix.
The preparation method: mixing and pulverizing the corn, the soybean meal, the stone powder, and the premix, and then mixing the mixture with the Haematococcus pluvialis algae powder and the vitamin E.

### Comparative Example 4 (same as Example 1)

The feed additive: 3 kg Haematococcus pluvialis algae powder, 0.89 kg vegetable oil, and 0.71 kg vitamin E.
The basal feed:
61.6 kg corn, 25.5 kg soybean meal, 7.2 kg stone powder, and 3.6 kg premix.
The preparation method: mixing and pulverizing the corn, the soybean meal, the stone powder, and the premix, and then mixing the mixture with the Haematococcus pluvialis algae powder, the vegetable oil, and the vitamin E.

### Experimental Example 1

120 laying hens were randomly selected and evenly divided into 4 groups (A, B, C, and D groups) with 30 laying hens in each group. The weight difference among the groups was not significant (P>0.05). The group A was fed with the basal feed; the group B was fed with the feed prepared in Example 1; the group C was fed with the feed prepared in Example 2; the group D was fed with the feed prepared in Example 3. The testing period was 42 days, and other feeding conditions were the same.

### Results

Under the same feeding conditions, after 6 days of feeding, the yolk color of the eggs of the B, C, and D groups was bright red compared with that of the yolk of the group A, and particularly, the yolk color of the eggs added with 3.0% Haematococcus pluvialis algae powder (group D) was more bright red. Therefore, after the laying hens had been fed with the feed provided by the present invention for 6 days, astaxanthin was enriched in the yolk, which increased the pigment content of the yolk and improved the nutrition value of egg products. When more than 3.0% of Haematococcus pluvialis algae powder was added, the cost was increased, but the color change of the yolk was not obvious.

The experimental result of the feeding comparative example shows that the yolk in the comparative example 4 (same as Example 1) with 3 kg Haematococcus pluvialis algae powder, 0.89 kg vegetable oil, and 0.71 kg vitamin E were bright red compared with the yolk of other ratios when the hens were fed for the same time under the same condition. Therefore, when Haematococcus pluvialis is added to the feed for feeding laying hens in the feed additive comparative examples provided by the present invention, adding a proper amount of vitamin E and vegetable oil can effectively dissolve astaxanthin, protect the astaxanthin from being oxidized, promote absorption of the laying hens, accelerate the enrichment of the astaxanthin in the yolk, increase the pigment content of the yolk, and further reduce the feeding cost.

The above descriptions are only preferred embodiments of the present invention. It should be noted that those of ordinary skill in the art can also make several improvements and modifications without departing from the principle of the present invention, and such improvements and modifications shall fall within the protection scope of the present invention.

What is claimed is:

1. A feed additive, comprising the following components in parts by weight: 5-50 parts of Haematococcus pluvialis, 6-10 parts of a vegetable oil, and 6-8 parts of vitamin E.

2. The feed additive according to claim 1, wherein the Haematococcus pluvialis is a cell wall-broken Haematococcus pluvialis.

3. The feed additive according to claim 1, wherein the Haematococcus pluvialis is a dry algae powder of a cell wall-broken Haematococcus pluvialis or an algae sludge of the cell wall-broken Haematococcus pluvialis.

4. The feed additive according to claim 3, wherein the Haematococcus pluvialis is 25-35 parts by weight.

5. A chicken feed, comprising the feed additive according to claim 1 and a basal feed.

6. The chicken feed according to claim 5, wherein a mass percentage of the feed additive in the chicken feed is 0.5-5%; a mass percentage of the basal feed is 93.4%-97.9%.

7. The chicken feed according to claim 6, wherein the basal feed comprises a corn, a soybean meal, a stone powder, and a premix; a mass ratio of the corn, the soybean meal, the stone powder, and the premix is (60-61.6):(24.9-25.5):(7-7.2):(3.5-3.6).

8. A method for preparing the chicken feed according to claim 5, comprising the following steps: pulverizing and mixing raw materials of the basal feed to obtain a resulting mixture, and then mixing the resulting mixture with the feed additive to obtain the chicken feed.

9. A method for producing astaxanthin eggs, comprising the following step: feeding laying hens with the chicken feed according to claim 5.

10. The method according to claim 9, wherein a feeding amount of the chicken feed is 0.05-0.15 g/day; a feeding time of the chicken feed is 3-42 days.

11. The feed additive according to claim 2, wherein the Haematococcus pluvialis is a dry algae powder of the cell wall-broken Haematococcus pluvialis or an algae sludge of the cell wall-broken Haematococcus pluvialis.

12. The chicken feed according to claim 5, wherein in the feed additive, the Haematococcus pluvialis is a cell wall-broken Haematococcus pluvialis.

13. The chicken feed according to claim 5, wherein in the feed additive, the Haematococcus pluvialis is a dry algae powder of a cell wall-broken Haematococcus pluvialis or an algae sludge of the cell wall-broken Haematococcus pluvialis.

14. The chicken feed according to claim 13, wherein in the feed additive, the Haematococcus pluvialis is 25-35 parts by weight.

15. The method according to claim 8, wherein a mass percentage of the feed additive in the chicken feed is 0.5-5%; a mass percentage of the basal feed is 93.4%-97.9%.

16. The method according to claim 15, wherein the basal feed comprises a corn, a soybean meal, a stone powder, and a premix; a mass ratio of the corn, the soybean meal, the stone powder, and the premix is (60-61.6):(24.9-25.5):(7-7.2):(3.5-3.6).

17. The method according to claim 9, wherein a mass percentage of the feed additive in the chicken feed is 0.5-5%; a mass percentage of the basal feed is 93.4%-97.9%.

18. The method according to claim 17, wherein the basal feed comprises a corn, a soybean meal, a stone powder, and a premix; a mass ratio of the corn, the soybean meal, the stone powder, and the premix is (60-61.6):(24.9-25.5):(7-7.2):(3.5-3.6).
